(19) United States
(12) Patent Application Publication
(10) Pub. No.: US 2018/0360771 A1
(43) Pub. Date: Dec. 20, 2018

(54) CANNABIDIOL CHEWING GUM

(71) Applicant: Yury Leon Shmerlis, Tampa, FL (US)

(72) Inventor: Yury Leon Shmerlis, Tampa, FL (US)

(21) Appl. No.: 15/622,576

(22) Filed: Jun. 14, 2017

(51) Int. Cl. A61K 31/05; A23G 4/06; A61K 9/68; A61K 31/12; A61K 31/522

(52) U.S. Cl. CPC A61K 31/05; A23G 4/068; A23G 4/06; A23V 2002/00; A61K 31/12; A61K 31/522; A61K 9/0058

(57) ABSTRACT

A cannabidiol chewing gum for delivering cannabidiol (CBD) to a user comprises one or more ingredients. The cannabidiol chewing gum comprises one or more of cannabidiol (CBD), gingerol, and caffeine. The user ingesting the cannabidiol chewing gum receives cannabidiol (CBD) from the cannabidiol chewing gum.

### TECHNICAL FIELD OF THE INVENTION

The invention disclosed herein generally relates to chewing gum. More particularly, the invention relates to a cannabidiol chewing gum for delivering cannabidiol (CBD) to a user.

### BACKGROUND

Cannabidiol (CBD) refers to one of at least 113 cannabinoids present in cannabis. Although cannabis are used as a recreational drug, the component cannabidiol (CBD) does not cause the “high” associated with the tetrahydrocannabinol (THC) component of cannabis. CBD, on the other hand, has been considered to have a wide scope of applications to multiple medical conditions. The scope is increased because CBD have little or no side effects and does not interfere with psychomotor learning and psychological functions of users. Furthermore, CBD has been used to treat certain forms of epilepsy. Typically, CBD constitutes up to forty percent of the extract from the cannabis plant. Existing methods of absorbing CBD require a user to smoke, vaporize, or ingest plant matter. Using these methods ensures the user ingests significant amounts of THC to generate hallucinogenic effects. Additionally, smoking affects the lungs of the user especially if the user has a related breathing condition.

Hence, there is a long felt but unresolved need for a method of ingesting cannabidiol (CBD), which eliminates the need to smoke, vaporize, or ingest cannabis plant matter.

### SUMMARY OF THE INVENTION

This summary is provided to introduce a selection of concepts in a simplified form that are further disclosed in the detailed description of the invention. This summary is not intended to identify key or essential inventive concepts of the claimed subject matter, nor is it intended for determining the scope of the claimed subject matter.

The cannabidiol chewing gum disclosed herein addresses the above-mentioned need for a method of ingesting cannabidiol (CBD), which eliminates the need to smoke, vaporize, or ingest cannabis plant matter. The cannabidiol chewing gum for delivering cannabidiol (CBD) to a user, disclosed herein, comprises one or more ingredients. The cannabidiol chewing gum comprises one or more of cannabidiol (CBD), gingerol, and caffeine. The user ingesting the cannabidiol chewing gum receives cannabidiol (CBD) from the cannabidiol chewing gum.

### DETAILED DESCRIPTION OF THE INVENTION

The present invention relates to a cannabidiol chewing gum for delivering cannabidiol (CBD) to a user, the cannabidiol chewing gum comprises one or more ingredients. The chewing gum core comprises one or more of cannabidiol (CBD), gingerol, and caffeine. The user ingesting the cannabidiol chewing gum receives cannabidiol (CBD) from the cannabidiol chewing gum. In an embodiment, the cannabidiol chewing gum comprises one or more flavors based on a preference of the user. In another embodiment, the cannabidiol chewing gum comprises artificial sweeteners based on a preference of the user. Moreover, the cannabidiol chewing gum may also comprise vitamins, antibiotics, colorants, etc. The cannabidiol chewing gum ensures delivery of cannabidiol (CBD) to the user who ingests it. The addition of vitamins or antibiotics provides added benefits to users having one or more medical conditions. When a user chews the cannabidiol chewing gum with cannabidiol (CBD), the absorption happens through the lining of the mouth without the harmful or hallucinogenic effects of smoking cannabis. Furthermore, this eliminates the need to smoke, vaporize, or ingest cannabis plant matter.

The foregoing examples have been provided merely for the purpose of explanation and are in no way to be construed as limiting of the cannabidiol chewing gum, disclosed herein. While the cannabidiol chewing gum has been described with reference to various embodiments, it is understood that the words, which have been used herein, are words of description and illustration, rather than words of limitation. Further, although the cannabidiol chewing gum, has been described herein with reference to particular means, materials, and embodiments, the cannabidiol chewing gum is not intended to be limited to the particulars disclosed herein; rather, the cannabidiol chewing gum extends to all functionally equivalent structures, methods and uses, such as are within the scope of the appended claims. Those skilled in the art, having the benefit of the teachings of this specification, may effect numerous modifications thereto and changes may be made without departing from the scope and spirit of the cannabidiol chewing gum disclosed herein in their aspects.

What is claimed is:

1. A cannabidiol chewing gum for delivering cannabidiol (CBD) to a user, the cannabidiol chewing gum comprising:
cannabidiol (CBD);
gingerol; and
caffeine
wherein ingesting the cannabidiol chewing gum delivers the cannabidiol (CBD) to the user.

2. The cannabidiol chewing gum of claim 1, further comprising:
one or more flavors.

3. The cannabidiol chewing gum of claim 1, further comprising:
one or more artificial sweeteners.
